(19) United States
(12) Patent Application Publication
(10) Pub. No.: US 2022/0387763 A1
(43) Pub. Date: Dec. 8, 2022

(54) CATHETER ASSEMBLY

(71) Applicant: TERUMO KABUSHIKI KAISHA, Tokyo (JP)

(72) Inventor: Masahiro ISHIDA, Kanagawa (JP)

(73) Assignee: TERUMO KABUSHIKI KAISHA, Tokyo (JP)

(21) Appl. No.: 17/888,440

(22) Filed: Aug. 15, 2022

Related U.S. Application Data
(63) Continuation of application No. PCT/JP2021/005953, filed on Feb. 17, 2021.

(30) Foreign Application Priority Data
Feb. 20, 2020 (JP) 2020-026892

(51) Int. Cl. A61M 25/06; A61M 25/00

(52) U.S. Cl. CPC A61M 25/0662; A61M 25/0014; A61M 25/0606; A61M 25/0097; A61M 25/0026

(57) ABSTRACT

A catheter assembly includes: a catheter; a catheter hub fixed to the catheter; an inner needle inserted through the catheter; a grip that fixes and holds the inner needle; and a support structure portion that contacts an outer peripheral surface of the catheter at a contact position to support an outer side of the catheter in an assembled state. In the assembled state, the support structure portion presses the catheter at the contact position without clamping the catheter at the contact position, and thereby deflects the catheter so that the catheter is curved along an extending direction when viewed from a direction orthogonal to the extending direction.

### CROSS-REFERENCE TO RELATED APPLICATIONS

This is a bypass continuation of PCT Application No. PCT/JP2021/005953, filed on Feb. 17, 2021, which claims priority to Japanese Application No. JP2020-026892, filed on Feb. 20, 2020. The contents of these applications are hereby incorporated by reference in their entireties.

### BACKGROUND

The present invention relates to a catheter assembly having a structure capable of supporting the outer side of a catheter at the time of puncture with the catheter and an inner needle.

When an introduction unit for infusion, blood transfusion, or the like is constructed in a treatment target (patient), a catheter assembly as disclosed in US 2016/0256667 A is used. The catheter assembly includes a multi-structure needle in which an inner needle is inserted into a catheter (outer needle). When the catheter assembly is used, a user punctures the multi-structure needle into a body of the treatment target, then causes the catheter to enter a blood vessel, and further removes the inner needle from the catheter to indwell the catheter.

During use, this type of catheter assembly is easily deflected when the multi-structure needle comes into contact with the treatment target, and this deflection makes it difficult for the multi-structure needle to be inserted into the body. Therefore, the catheter assembly disclosed in US 2016/0256667 A has a structure in which a distal end of a grip fixing the inner needle is arranged at a position near the outer periphery of the catheter to support the catheter at the time of puncture.

### SUMMARY

However, if the support structure portion of the catheter assembly strongly clamps the outer side of the catheter in order to suppress the deflection of the catheter, the sliding resistance applied to the catheter increases, and it is difficult for the user to operate the catheter to move. Therefore, the conventional support structure portion is formed such that there is a clearance with respect to the catheter in an assembled state of the catheter assembly.

However, the support structure portion formed in this manner increases the clearance with respect to an outer peripheral surface of the catheter when the catheter is manufactured to be thin within a range of a dimensional tolerance, for example. As a result, there is a disadvantage that it becomes difficult for the support structure portion to effectively support the catheter at the time of puncture with the multi-structure needle.

The present invention relates to the technique of the catheter assembly described above, and an object thereof is to provide a catheter assembly capable of appropriately supporting a catheter to suppress deflection at the time of puncture and allowing for easy movement of the catheter when operated by a user.

According to one embodiment of the disclosure, a catheter assembly includes: a catheter; a catheter hub fixed to the catheter; an inner needle inserted through the catheter; a grip that fixes and holds the inner needle; and a support structure portion that contacts an outer peripheral surface of the catheter at a contact position to support an outer side of the catheter in an assembled state. In the assembled state, the support structure portion presses the catheter at the contact position without clamping the catheter at the contact position, and thereby deflects the catheter so that the catheter is curved along an extending direction when viewed from a direction orthogonal to the extending direction.

The above-described catheter assembly can appropriately support the catheter to suppress the deflection at the time of puncture, and allows for easy movement of the catheter when operated by the user.

### BRIEF DESCRIPTION OF THE DRAWINGS

FIG. 1 is a perspective view of a catheter assembly according to a first embodiment of the present invention;

FIG. 2 is an exploded perspective view of the catheter assembly of FIG. 1;

FIG. 3 is a perspective view of a catheter operation member as viewed from obliquely below;

FIG. 4 is a side sectional view of a distal portion of the catheter assembly;

FIG. 5 is a partial front sectional view illustrating a distal portion of the catheter assembly;

FIG. 6A is a side sectional view schematically illustrating an assembled state of a catheter and a support structure portion; FIG. 6B is a side sectional view schematically illustrating an assembled state of a catheter and a support structure portion of the related art;

FIG. 7A is a side sectional view schematically illustrating an assembled state of a catheter and a support structure portion according to a first modification; FIG. 7B is a side sectional view schematically illustrating an assembled state of a catheter and a support structure portion according to a second modification; FIG. 7C is a side sectional view schematically illustrating an assembled state of a catheter and a support structure portion according to a third modification;

FIG. 8 is a side sectional view schematically illustrating an assembled state of a catheter and a support structure portion according to a fourth modification;

FIG. 9A is a side view illustrating a state of the catheter assembly at the time of puncture; FIG. 9B is a side view illustrating a state in which the catheter operation member is advanced after puncture;

FIG. 10A is a side sectional view schematically illustrating a supporting state of the catheter at the time of puncture; FIG. 10B is a side sectional view schematically illustrating a supporting state of the catheter when the catheter is greatly deflected;

FIG. 11 is a partial front sectional view illustrating a distal portion of a catheter assembly according to a second embodiment;

FIG. 12 is a plan sectional view along an axial center of a catheter in the catheter assembly of FIG. 11;

FIG. 13A is a plan sectional view schematically illustrating an assembled state of the catheter and a support structure portion; FIG. 13B is a plan sectional view schematically illustrating the supporting state of the catheter when the catheter is greatly deflected; FIG. 13C is a plan sectional view schematically illustrating an assembled state of a catheter and a support structure portion according to a fifth modification;

FIG. 14A is a plan sectional view schematically illustrating an assembled state of a catheter and a support structure portion according to a sixth modification; FIG. 14B is a plan sectional view schematically illustrating an operation of a lower support member when the catheter of FIG. 14A moves;

FIG. 15 is a perspective view illustrating a distal portion of a catheter assembly according to a third embodiment;

FIG. 16A is a plan sectional view schematically illustrating an assembled state of a catheter and a support structure portion of FIG. 15; FIG. 16B is a plan sectional view schematically illustrating a supporting state of the catheter when the catheter of FIG. 16A is greatly deflected;

FIG. 17A is a plan sectional view schematically illustrating an assembled state of a catheter and a support structure portion according to a seventh modification; and FIG. 17B is a plan sectional view schematically illustrating a supporting state of the catheter when the catheter of FIG. 17A is greatly deflected.

### DETAILED DESCRIPTION

Hereinafter, preferred embodiments of the present invention will be described in detail with reference to the accompanying drawings.

### First Embodiment

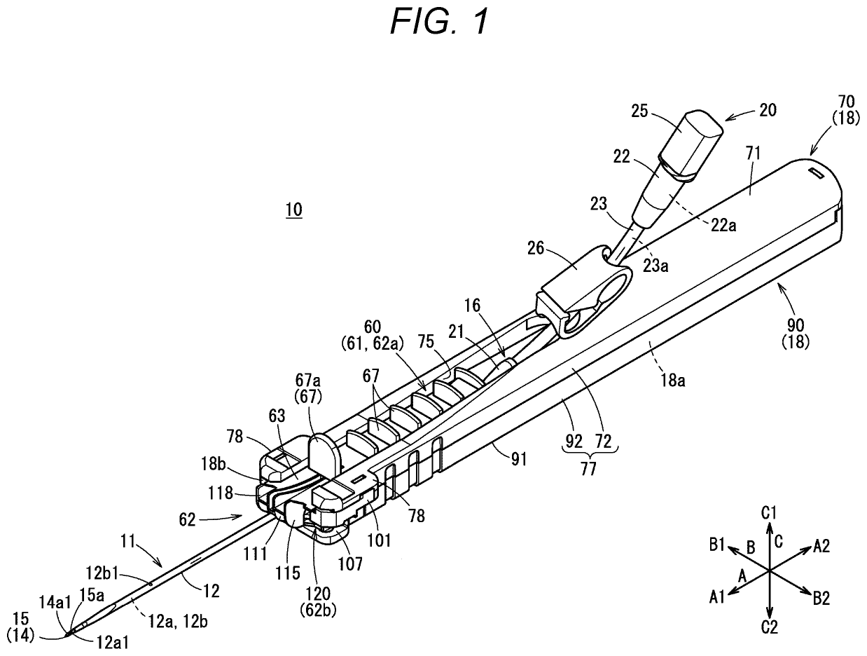

As illustrated in FIG. 1, a catheter assembly 10 according to a first embodiment of the present invention is a medical device used at the time of performing infusion, blood transfusion, blood sampling, or the like on a treatment target (living body), and a catheter 12 is inserted into and indwelled inside the body of the treatment target to cause the inside and outside of the body to communicate. This catheter assembly 10 enables insertion of a catheter 12 having a longer length (for example, a central venous catheter, a PICC, a mid-line catheter, and the like) than a peripheral venous catheter. Incidentally, the catheter assembly 10 may also be configured to enable insertion of the peripheral venous catheter. In addition, the catheter assembly 10 is not limited to the venous catheter, and may be configured to enable insertion of an arterial catheter such as a peripheral arterial catheter.

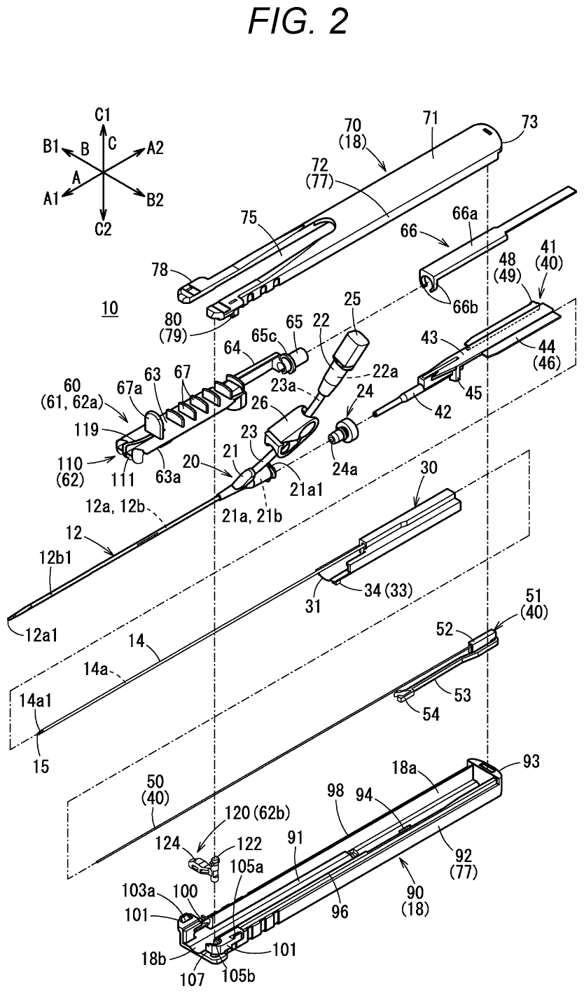

As illustrated in FIGS. 1 and 2, the catheter assembly 10 includes an inner and outer needle assembly 16 in which the catheter 12, an inner needle 14, a catheter hub 20, an inner needle hub 30, a safety member 40, and a catheter operation member 60 are assembled in an assembled state before use (before puncture). Further, the catheter assembly 10 includes a grip 18 (housing) configured to accommodate the inner and outer needle assembly 16 and be gripped by a user.

In the inner and outer needle assembly 16 in the assembled state, the inner needle 14 penetrates through the catheter 12 and the catheter hub 20, and a needle tip 15 of the inner needle 14 forms a multi-structure needle 11 protruding from a distal end of the catheter 12. The safety member 40 through which the inner needle 14 is inserted is arranged on the proximal side of the catheter hub 20, and the inner needle hub 30 holding the inner needle 14 is arranged on the proximal side of the safety member 40. The catheter operation member 60 is arranged above the catheter 12, the catheter hub 20, and the safety member 40, and causes advancement and retraction of these members under the operation of the user. The inner and outer needle assembly 16 including a portion of the multi-structure needle 11 on the proximal side is accommodated in the grip 18, and the inner needle hub 30 is fixed to the grip 18.

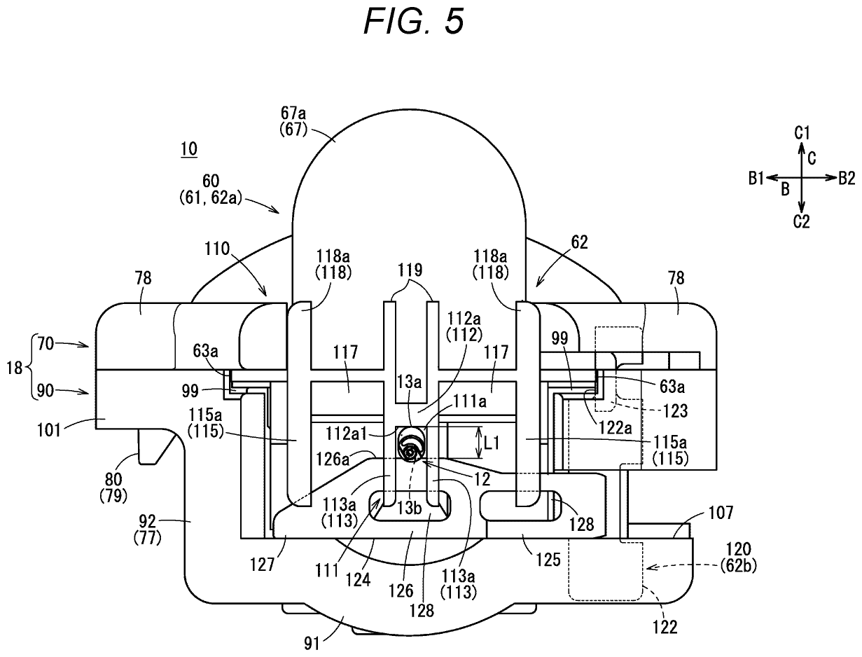

The catheter 12 according to the present embodiment is a tubular body having appropriate flexibility, and is configured as a multi-lumen type having a plurality of lumens 12a and 12b therein (see also FIG. 5). The lumens 12a and 12b extend in the axial direction (arrow A direction) of the catheter 12, and communicate with a distal opening 12a1 and a distal opening 12b1, respectively, at the distal end of the catheter 12. For example, the lumen 12a is formed in a circular shape capable of accommodating the inner needle 14, and the lumen 12b is formed in an arc-shaped ellipse above the lumen 12a. A length of the catheter 12 is set within a range of about 14 to 500 mm, preferably within a range of 30 to 400 mm, and more preferably within a range of 76 to 200 mm.

A constituent material of the catheter 12 is a soft resin material is suitable, and examples thereof include a fluorine-based resin such as polytetrafluoroethylene (PTFE), ethylene-tetrafluoroethylene copolymer (ETFE) and perfluoroalkoxy fluorine resin (PFA), an olefin-based resin such as polyethylene and polypropylene or a mixture thereof, polyurethane, polyester, polyamide, polyether nylon resin, a mixture of the olefin-based resin and ethylene-vinyl acetate copolymer, and the like. Note that it is a matter of course that the catheter 12 may be a single lumen type including only the lumen 12a through which the inner needle 14 is inserted without being limited to the multi-lumen type in the present invention.

A proximal end of the catheter 12 is fixed to a distal end in the catheter hub 20 by an appropriate fixing means such as caulking, fusion, and adhesion. The catheter hub 20 is exposed on a skin of a treatment target in a state in which the catheter 12 has been inserted into a blood vessel of the treatment target, and indwells together with the catheter 12 by being pasted with a tape or the like.

The catheter hub 20 has two separate hubs (main hub 21 and sub hub 22) corresponding to the multi-lumen type catheter 12. The main hub 21 is a member directly connected to the catheter 12, and the sub hub 22 is a member connected to the main hub 21 through a tube 23.

A constituent material of the catheter hub 20 (main hub 21 and sub hub 22) is not particularly limited, but a thermoplastic resin such as polypropylene, polycarbonate, polyamide, polysulfone, polyarylate, and a methacrylate-butylene-styrene copolymer may be preferably applied, for example.

The main hub 21 is a tubular body extending in parallel with the axis on the proximal side of the catheter 12, and the tube 23 is connected to a predetermined position on an outer peripheral surface of the main hub 21. An internal space 21a communicating with the lumen 12a and an internal space 21b communicating with the lumen 12b are provided inside the main hub 21. A proximal end of the internal space 21a communicates with a proximal opening 21a1 of the main hub 21. On the other hand, the internal space 21b is separated from the internal space 21a and communicates with a lumen 23a of the tube 23 inserted into and fixed to the main hub 21.

In the assembled state, a valve member 24 is inserted into the main hub 21 from the proximal opening 21a1 toward the back side (arrow A1 side) of the internal space 21a. A valve hole 24a that can be elastically opened and closed is provided at the axial center of the valve member 24. Distal ends of the inner needle 14 and the safety member 40 are inserted into the valve hole 24a in the assembled state of the valve member 24, so that an inner surface of the valve hole 24a and an outer surface of the safety member 40 are in close contact with each other. As a result, the valve member 24 brings the catheter hub 20 and the safety member 40 into a fitted state, and prevents leakage of blood from the proximal opening 21a1 of the main hub 21 at the time of puncture with the inner needle 14.

In addition, the sub hub 22 is formed in a tubular shape having the same thickness as the main hub 21, and a proximal end of the tube 23 is inserted from a distal end of the sub hub 22 and fixed thereto. An internal space 22a communicating with the lumen 23a of the tube 23 is formed inside the sub hub 22. A proximal end of the internal space 22a communicates with a proximal opening (not illustrated) of the sub hub 22. In the assembled state, a closing member 25 that closes the proximal opening is connected to the sub hub 22.

The tube 23 is configured to have flexibility similarly to the catheter 12. A clamp 26 capable of opening and closing the lumen 23a of the tube 23 is attached in advance to an intermediate position in the extending direction of the tube 23.

Meanwhile, the inner needle 14 of the catheter assembly 10 is configured as a hollow tubular body having rigidity capable of puncturing a skin of a living body. The needle tip 15, which is sharp, is provided at the distal end of the inner needle 14. The needle tip 15 has a blade surface 15a inclined toward the distal side and the upper side. That is, the blade surface 15a faces upward (toward an arrow C1 side and the catheter operation member 60 side). A hollow portion 14a is formed inside the inner needle 14 to penetrate along the arrow A direction, and the hollow portion 14a communicates with a distal opening 14a1 provided in the blade surface 15a.

Examples of a constituent material of the inner needle 14 include a metal material such as stainless steel, aluminum or an aluminum alloy, and titanium or a titanium alloy, a hard resin, ceramics, and the like. The inner needle 14 is firmly fixed to the inner needle hub 30 by an appropriate fixing means such as fusion, adhesion, and insert molding.

The inner needle hub 30 is a member configured to directly hold the inner needle 14 and is fixed to the grip 18 through a grip fixing portion 31 (lower wall) formed on an arrow C2 side. The grip fixing portion 31 has a lower surface provided with a plurality of fixing protrusions 34 that protrude short in the downward direction and constitute an attachment mechanism 33 with the grip 18.

The safety member 40 is configured to be inserted and fitted into the main hub 21 and the valve member 24 of the catheter hub 20 to follow the catheter hub 20 that is moving. The safety member 40 includes a cover body 41 that covers the outer side of the inner needle 14 along with advancement, a blunt needle 50 protruding from the needle tip 15 of the inner needle 14 after puncture, and the blunt needle hub 51 holding the blunt needle 50.

The cover body 41 includes: a distal cover portion 42 that has a cylindrical shape and accommodates and protects the inner needle 14 after puncture; a proximal flat plate portion 43 extending from an upper portion of the distal cover portion 42 to the arrow A2 side; and a pair of protruding pieces 44 protruding outward in the width direction from the proximal flat plate portion 43. In addition, an engagement projection 45 with which the blunt needle hub 51 is engaged is provided at a point where the proximal flat plate portion 43 and the distal cover portion 42 are connected.

The distal cover portion 42 is inserted into and brought into close contact with the valve member 24 on the distal side, thereby being frictionally fitted to the catheter hub 20 including the valve member 24. In the assembled state, a proximal end of the distal cover portion 42 faces a distal end of the inner needle hub 30. The proximal flat plate portion 43 connected to the distal cover portion 42 extends to a proximal end in the grip 18 along an upper portion of the inner needle hub 30 in the assembled state.

The pair of protruding pieces 44 protrudes outward in the width direction (arrow B direction) from the inner needle hub 30 and extends to the vicinity of a side wall 77 of the grip 18 (above rail walls 96 and 98 to be described later). The respective protruding pieces 44 constitute a guide mechanism 46 which performs guide in the arrow A direction in cooperation with the grip 18 when the safety member 40 moves. In addition, a locked protrusion 48 is provided on a side of the protruding piece 44 on an arrow B1 side. The locked protrusion 48 is locked to a locking portion 100 of the grip 18 at the advanced position at which the safety member 40 has advanced, thereby constituting one of safety movement restricting mechanisms 49 that restrict the advancement and retraction of the cover body 41.

The blunt needle 50 of the safety member 40 is a rod member (round rod) configured to prevent erroneous puncture of the inner needle 14 with respect to a catheter 12 and a living body, and is movably accommodated in the hollow portion 14a of the inner needle 14. A distal end of the blunt needle 50 is formed in a shape blunter than the needle tip 15 (for example, to have a polished flat surface), and is arranged at a position near a proximal end of the distal opening 14a1 of the inner needle 14 in the assembled state. The distal end of the blunt needle 50 is exposed from the needle tip 15 (distal opening 14a1) as the safety member 40 advances.

The blunt needle hub 51 is configured to be relatively movable with respect to the inner needle 14, the inner needle hub 30, and the grip 18 by holding the blunt needle 50 and engaging with the engagement projection 45 of the cover body 41. The blunt needle hub 51 includes a blunt needle holding portion 52 that holds the blunt needle 50 on the arrow A2 side, and an arm portion 53 extending from the blunt needle holding portion 52 to the arrow A1 side.

The blunt needle holding portion 52 is arranged in a space on the proximal side of a point where the inner needle 14 is fixed in the inner needle hub 30. When a distal end surface of the blunt needle holding portion 52 comes into contact with an inner needle fixing block (not illustrated) of the inner needle hub 30 as the blunt needle hub 51 advances, the subsequent advancement of the blunt needle hub 51 is prevented.

The entire extending portion of the arm portion 53 is configured to be elastically deformable in the width direction, and an engagement end 54 to be engaged with the engagement projection 45 in the assembled state is provided at the distal end of the arm portion 53. When the cover body 41 further advances at a stage where the movement of the blunt needle hub 51 is prevented, the engagement end 54 is elastically deformed as appropriate to release the engagement with the engagement projection 45.

Note that the safety member 40 is not limited to the above configuration as long as erroneous puncture of the needle tip 15 of the inner needle 14 can be prevented. For example, the safety member 40 may include only the cover body 41 without including the blunt needle 50 and the blunt needle hub 51.

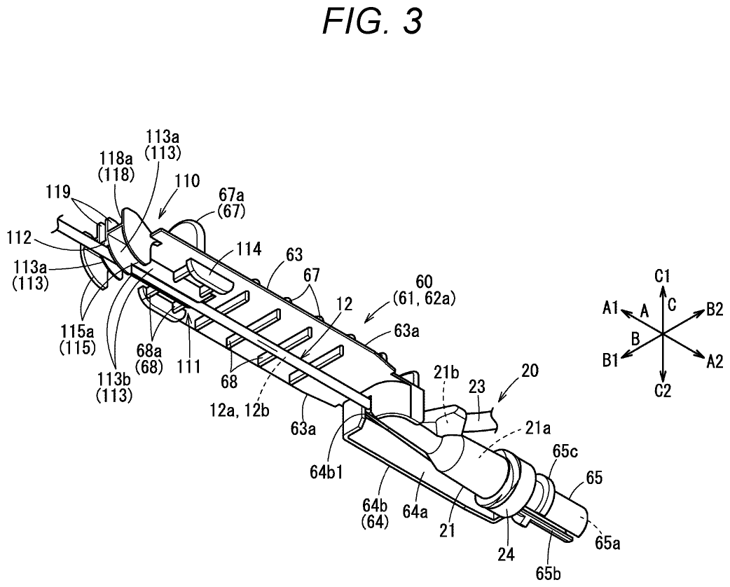

As illustrated in FIGS. 2 and 3, the catheter operation member 60 constitutes an operation unit 61 to be operated by the user in the catheter assembly 10. In addition, the catheter operation member 60 according to the present embodiment is also a first support portion 62a of a support structure portion 62 that supports the outer side of the catheter 12 (multi-structure needle 11) at the time of puncture with the multi-structure needle 11. A material forming the catheter operation member 60 is not particularly limited, and, for example, the materials exemplified for the catheter hub 20 can be appropriately selected.

Specifically, the catheter operation member 60 includes: an operation plate portion 63 (extending portion) extending in the arrow A direction; a hub engagement portion 64 connected to a proximal end of the operation plate portion 63 and engaged with the catheter hub 20; and an operation member tubular portion 65 which is connected to a proximal end of the hub engagement portion 64 and accommodates the safety member 40. In addition, the catheter operation member 60 includes a cover member 66 which extends in the proximal direction from the hub engagement portion 64 and covers the safety member 40.

The operation plate portion 63 is a site in contact with a user's finger to perform an advancing/retracting operation. The operation plate portion 63 is formed to be thin and has flexibility capable of being curved in a direction away from the multi-structure needle 11. The operation plate portion 63 has side edges 63a, which extend in the arrow A direction, on both sides in the width direction. A plurality of tabs 67 are provided on an upper surface of the operation plate portion 63. A tab 67a at the most distal end among the plurality of tabs 67 protrudes more than the other tabs 67. Further, a plurality of ribs 68 protrude short from a lower surface of the operation plate portion 63. The catheter 12 is arranged below the plurality of ribs 68.

Then, an operation unit support region 110 configured to support the catheter 12 is provided on the distal side of the operation plate portion 63. The operation unit support region 110 constitutes a portion of the operation plate portion 63 where a deflection is restricted. The operation unit support region 110 includes an upper support portion 111, which covers the catheter 12 (multi-structure needle 11) from above, on the lower surface of the operation plate portion 63.

The upper support portion 111 is provided at a central portion of the catheter operation member 60 in the width direction and extends from the distal end to the arrow A2 side by a predetermined length (over a range of the operation unit support region 110). A proximal end of the upper support portion 111 is located on the arrow A2 side with respect to the tab 67a. The upper support portion 111 includes: a base portion 112 that can come into contact with an arrow C1 side of the catheter 12; and a pair of ridge portions 113 that can come into contact with the catheter 12 in the arrow B direction.

The base portion 112 protrudes to a position shifted downward (to the arrow C2 side) with respect to the operation plate portion 63 and extends along the extending direction (arrow A direction) of the catheter 12. A distal end surface of the base portion 112 is flush and continuous with edge portions of the pair of ridge portions 113. The base portion 112 is molded to be thicker than the operation plate portion 63, and suppresses a deflection of the operation unit support region 110 along a vertical direction (arrow C direction).

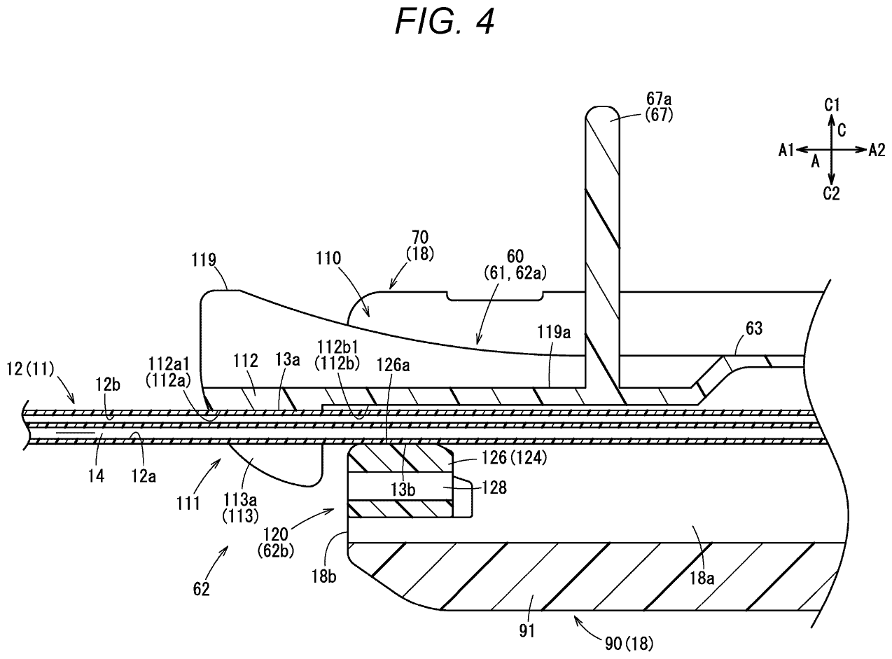

Then, the base portion 112 is formed in a stepped shape having a plurality of different lower surface positions (a distal projecting portion 112a and a proximal extending portion 112b) along the arrow A direction in a side sectional view illustrated in FIG. 4. That is, the distal projecting portion 112a is at a position protruding downward (to the arrow C2 side) from the proximal extending portion 112b. A difference in protruding amount of the distal projecting portion 112a with respect to the proximal extending portion 112b is slight, and is set to, for example, 1/10 or less of a protruding amount from the operation plate portion 63 to the distal projecting portion 112a.

Each of a lower surface 112a1 of the distal projecting portion 112a and a lower surface 112b1 of the proximal extending portion 112b extends linearly along the arrow A direction. A length of the distal projecting portion 112a in the arrow A direction is shorter than a length of the proximal extending portion 112b in the arrow A direction. The distal projecting portion 112a is provided in a range that exactly overlaps a distal projecting piece 113a of each of the ridge portions 113 to be described later in a side view.

The distal projecting portion 112a described above is arranged on the distal side of the grip 18 (a lower support member 120 to be described later) in the assembled state of the catheter assembly 10. The lower surface 112a1 of the distal projecting portion 112a is in contact with an outer peripheral surface of the catheter 12 to constitute a portion that presses the catheter 12. On the other hand, the proximal extending portion 112b faces the outer peripheral surface of the catheter 12 in a non-contact manner (forms a clearance with respect to the catheter 12).

As illustrated in FIGS. 3 and 4, the pair of ridge portions 113 of the upper support portion 111 is configured to sandwich the base portion 112 therebetween, and protrudes from the lower surface of the operation plate portion 63 to the arrow C2 side. A protruding amount of each of the ridge portions 113 from the operation plate portion 63 is sufficiently larger than a protruding amount of the base portion 112. In the assembled state of the catheter assembly 10, the respective ridge portions 113 are designed with an interval at positions not in contact with the outer peripheral surface of the catheter 12 (or in contact with the catheter 12 to the extent not to deflect the catheter 12).

In addition, each of the ridge portions 113 is formed to have a step along the arrow A direction similarly to the stepped shape (the distal projecting portion 112a and the proximal extending portion 112b) of the base portion 112. The distal side (distal projecting piece 113a) of each of the ridge portions 113 is sufficiently longer toward the arrow C2 side than an arrangement position of the catheter 12. An edge portion of each of the distal projecting pieces 113a is curved toward the proximal side (arrow A2 side) from a distal end connected to the operation plate portion 63 to the arrow C2 side, and linearly extends to the arrow C1 side at a protruding top on the arrow C2 side.

A lower edge portion on the proximal side (a proximal projecting piece 113b) of each of the ridge portions 113 linearly extends from a point, connected to the edge portion of the distal projecting piece 113a, to the arrow A2 side. Each of the proximal projecting pieces 113b protrudes from the base portion 112 by a length substantially equal to an outer diameter of the catheter 12. Some ribs 68a on the distal side among the plurality of ribs 68 provided on the lower surface of the operation plate portion 63 are provided to be continuous with the respective proximal projecting pieces 113b. These ribs 68a are continuous with side ribs 114 extending short in the arrow A direction on the outer side in the width direction. The side rib 114 protrudes to the arrow C2 side more than the rib 68a, and has a function of guiding the advancement and retraction of the catheter operation member 60 with respect to the grip 18.

In the upper support portion 111 described above, the catheter 12 is arranged in a support space 111a formed by the base portion 112 and the pair of ridge portions 113 in the assembled state. That is, the catheter 12 is covered by the base portion 112 on the upper side and covered by the pair of ridge portions 113 on the right and left sides in the width direction.

In addition, the operation unit support region 110 includes a pair of lower protruding blocks 115 protruding toward the arrow C2 side on the lower surface side of the operation plate portion 63 and on the outer side of the upper support portion 111 in the width direction. Each of the lower protruding blocks 115 is provided with a predetermined gap 117 with respect to the upper support portion 111.

The pair of lower protruding blocks 115 also protrudes toward the arrow C2 side to the same extent as the distal projecting pieces 113a of the pair of ridge portions 113. In addition, a width (thickness) of each of the lower protruding blocks 115 is larger than a width (thickness) of the ridge portion 113. A distal end surface 115a of each of the lower protruding blocks 115 is curved to the arrow A2 side as proceeding to the arrow C2 side.

That is, each of the distal projecting pieces 113a and each of the lower protruding blocks 115 are at positions overlapping each other in a side view, and are formed in substantially the same shape. As a result, the distal side and the lower side (the arrow A1 side and the arrow C2 side) of the catheter operation member 60 are guided such that the catheter operation member 60 is separated from the catheter 12 when the catheter operation member 60 is delivered from the grip 18 to some extent and comes into contact with a body surface of the treatment target.

Further, the operation unit support region 110 has a pair of upper protruding blocks 118, which protrude short toward the arrow C1 side at the same position as the lower protruding block 115 in the width direction on the upper surface side of the operation plate portion 63. A width of each of the upper protruding blocks 118 coincides with the width of each of the lower protruding blocks 115. A distal end surface 118a of each of the upper protruding blocks 118 is flush with an upper end of the distal end surface 115a of the lower protruding block 115.

Further, a pair of reinforcing pieces 119 (reinforcing portions) is provided on the inner side of the pair of upper protruding blocks 118 in the width direction. Each of the reinforcing pieces 119 is provided at the same position as each of the ridge portions 113 in the width direction and protrudes to the opposite side (arrow C1 side) of each of the ridge portions 113. Each of the reinforcing pieces 119 protrudes from the upper surface of the operation plate portion 63 to the same extent as the upper protruding block 118 at its distal end, is formed to be gradually lowered while being curved from this distal end to the arrow A2 side, and is in contact with the tab 67a. Since the operation plate portion 63 does not exist between the respective reinforcing pieces 119, a groove portion 119a in which an upper surface of the base portion 112 of the upper support portion 111 is exposed is formed.

Meanwhile, the hub engagement portion 64 continuous with the proximal end of the operation plate portion 63 has an accommodation chamber 64a for accommodating the main hub 21, and has a wall portion 64b on the arrow B1 side to be formed in a shape in which the arrow B2 side is cut out (shape in which the accommodation chamber 64a is opened). This shape is formed to expose the sub hub 22 and the tube 23 of the catheter hub 20 configured as the multi-lumen type. The distal side of the hub engagement portion 64 extends such that the wall portion 64b on the arrow B1 side wraps around the accommodation chamber 64a. The wall portion 64b on the distal side is provided with a gap 64b1 which is narrower than a diameter of the catheter hub 20 and from which only the catheter 12 (multi-structure needle 11) extends.

The operation member tubular portion 65 is formed in a cylindrical shape that protrudes short in the proximal direction from a proximal end of the hub engagement portion 64. A communicating space 65a where the safety member 40 (cover body 41) is inserted and arranged is provided on the inner side the operation member tubular portion 65. In addition, a slit 65b communicating with the accommodation chamber 64a and the communicating space 65a is formed in the lower portion of the operation member tubular portion 65. An arcuate rib 65c that protrudes in the circumferential direction and restricts the movement of the cover member 66 is provided on the outer peripheral surface of the operation member tubular portion 65.

Returning to FIG. 2, the cover member 66 is mounted on the catheter operation member 60 and is provided to prevent the user from directly coming into contact with the safety member 40. The cover member 66 includes a body portion 66a that covers the upper side (side on which a hand is located when being gripped by the user) of the safety member 40, and a pair of attachment leg portions 66b provided at a distal end of the body portion 66a and attached to the operation member tubular portion 65. The pair of attachment leg portions 66b is engaged between the proximal end surface of the hub engagement portion 64 and the arcuate rib 65c.

In addition, the grip 18 of the catheter assembly 10 is formed to have an appropriate thickness that can be easily held by the user, and extends along the arrow A direction. The accommodation space 18a in which the catheter 12, the catheter hub 20, the safety member 40, and the catheter operation member 60 can advance and retract is formed in the grip 18. The accommodation space 18a communicates with a distal opening portion 18b of the grip 18. The grip 18 is configured by assembling an upper grip 70 and a lower grip 90, which can be divided in the arrow C direction, with each other.

The upper grip 70 has a ceiling wall 71, a pair of upper side walls 72, and an upper rear wall 73, and is formed in a recessed shape (bowl shape) that is opened downward. The pair of upper side walls 72 constitutes side walls 77 on both sides in the width direction of the grip 18 together with a lower side wall 92 of the lower grip 90.

In addition, the ceiling wall 71 has an operation member exposure notch 75 at the center in the arrow B direction closer to the distal side than an intermediate portion in the arrow A direction. The operation member exposure notch 75 is opened at the distal end to communicate with the accommodation space 18a, and exposes the tube 23 of the catheter hub 20 and the tab 67 of the catheter operation member 60 so as to be capable of advancing and retracting. Further, the upper grip 70 has a pair of upper projecting pieces 78 at the distal end, and a fixing hook 80 constituting a fixing mechanism 79 between the upper grip 70 and the lower grip 90 is provided on lower surfaces of the respective upper projecting pieces 78 and the upper rear wall 73.

The lower grip 90 has a bottom wall 91, a pair of lower side walls 92, and a lower rear wall 93, and is formed in a recessed shape (bowl shape) opened upward. A predetermined range on the arrow A2 side of the bottom wall 91 is a mounting target portion on which the inner needle hub 30 is mounted, and a plurality of (three in the present embodiment) mounting holes 94 (a part of the attachment mechanism 33) into which the fixing protrusion 34 of the inner needle hub 30 can be fitted are provided.

In addition, the pair of lower side walls 92 has the rail walls 96 and 98 in the upper portion thereof, and the upper side wall 72 of the upper grip 70 is arranged on the outer side of the rail walls 96 and 98 in the width direction in an assembled state. In the assembled state, the pair of side edges 63a of the catheter operation member 60 and the protruding pieces 44 of the safety member 40 are slidably arranged on the pair of rail walls 96 and 98. In addition, the locked protrusion 48 of the cover body 41 is arranged between the upper side wall 72 on the arrow B1 side and the rail wall 98.

The lower side wall 92 on the arrow B1 side is provided with a locking portion 100 which is engaged with the locked protrusion 48 to define an advancement limit of the cover body 41 and restrict the retraction of the cover body 41 when the cover body 41 of the safety member 40 advances. That is, the locking portion 100 constitutes the safety movement restricting mechanism 49 together with the locked protrusion 48. The catheter assembly 10 can reliably cover (protect) the inner needle 14 after puncture with the cover body 41 by restricting detachment of the safety member 40 from the grip 18.

A pair of projecting bodies 101 protruding outward in the width direction from the pair of lower side walls 92 is provided at the distal end of the lower grip 90. The projecting body 101 on the arrow B1 side has a first fixing hole 103a that is a part of the fixing mechanism 79. The projecting body 101 on the arrow B2 side has a second fixing hole 105a that is a part of the fixing mechanism 79. In addition, the projecting body 101 on the arrow B2 side has a movement space 107 in which the lower support member 120 is rotatable.

As illustrated in FIGS. 2, 4, and 5, the lower support member 120 is rotatably attached to the grip 18, and constitutes a second support portion 62b of the support structure portion 62 that supports the catheter 12 (multi-structure needle 11) extending below the catheter operation member 60 from below in the assembled state. In addition, the lower support member 120 rotates by contact with the wall portion 64b of the hub engagement portion 64 when the catheter operation member 60 advances, and enables the catheter operation member 60 (and the catheter hub 20 and the safety member 40) to be delivered from the accommodation space 18a.

The lower support member 120 includes a shaft 122 extending in the arrow C direction and a lower support portion 124 protruding in a direction orthogonal to the axial center of the shaft 122. The shaft 122 has an upper portion provided with a guide plane 122a continuous with an upper end of the rail wall 96 and a pair of small projections 123 flush with the guide plane 122a. The side edge 63a of the catheter operation member 60 is close to the guide plane 122a in the assembled state. As a result, the rotation of the lower support member 120 is restricted in a state in which the side edge 63a is present.

The lower support portion 124 has an appropriate thickness in the arrow C direction, and is connected to the lower side (arrow C2 side) of an intermediate position of the shaft 122 in the arrow C direction. The lower support portion 124 is formed to be thicker than a thickness of the bottom wall 91 of the lower grip 90, for example. The lower support portion 124 is formed by providing a coupling portion 125 connected to the shaft 122, a central portion 126 located at the central portion in the width direction, and a protruding end 127 located at a position farthest from the shaft 122 to be continuous with each other from the arrow B2 side to the arrow B1 side.

The coupling portion 125 and the central portion 126 are provided respectively with lightening holes 128 penetrating in the arrow A direction in order to enhance the molding accuracy of the lower support portion 124. The central portion 126 slightly protrudes upward (to the arrow C1 side) from the coupling portion 125 and the protruding end 127, and has the largest thickness in the lower support portion 124. The lower support portion 124 basically supports the lower side of the catheter 12 at the central portion 126. An upper surface of the protruding end 127 is gradually inclined downward from the central portion 126 to the arrow B1 side.

The lower support member 120 is inserted along a bearing notch 105b from above the lower grip 90 in a posture in which the lower support portion 124 faces the arrow B1 side. At this time, the lower support member 120 is smoothly inserted into the bearing notch 105b as the vicinity of the coupling portion of the lower support portion 124 passes through an opening portion of the bearing notch 105b on the accommodation space 18a side. When the upper grip 70 and the lower grip 90 are mounted, the upper end of the shaft 122 supported by the lower grip 90 is pivotally supported by the upper grip 70.

In the assembled state of the lower support member 120, the side edge 63a of the catheter operation member 60 is present on the guide plane 122a, and thus, the lower support portion 124 is restricted from rotating and stands by to be capable of supporting the catheter 12. As a result, the lower support portion 124 supports the catheter 12 from below to suppress the deflection of the catheter 12. When the catheter operation member 60 advances from the grip 18, the lower support member 120 becomes rotatable as the side edge 63a comes out of the guide plane 122a, and allows the catheter hub 20, the catheter operation member 60, and the safety member 40 to be delivered.

In the catheter assembly 10 described above, the inner and outer needle assembly 16 (catheter 12, inner needle 14, catheter hub 20, inner needle hub 30, safety member 40, and catheter operation member 60) is assembled first at the time of assembly. Thereafter, the inner and outer needle assembly 16 is arranged on the lower grip 90 incorporating the lower support member 120, and the upper grip 70 is mounted on the inner and outer needle assembly 16 and the lower grip 90.

As illustrated in FIG. 5, the side edge 63a of the catheter operation member 60 is arranged in a guide space 99 of the grip 18 (between the upper grip 70 and the lower grip 90) in the assembled state of the catheter assembly 10. As a result, the catheter operation member 60 is prevented from moving or deflecting in the arrow C direction (vertical direction) inside the grip 18. The support space 111a of the support structure portion 62 is surrounded by the base portion 112, the pair of ridge portions 113, and the lower support portion 124 to form a quadrangle shape (substantially a square shape) in a front sectional view.

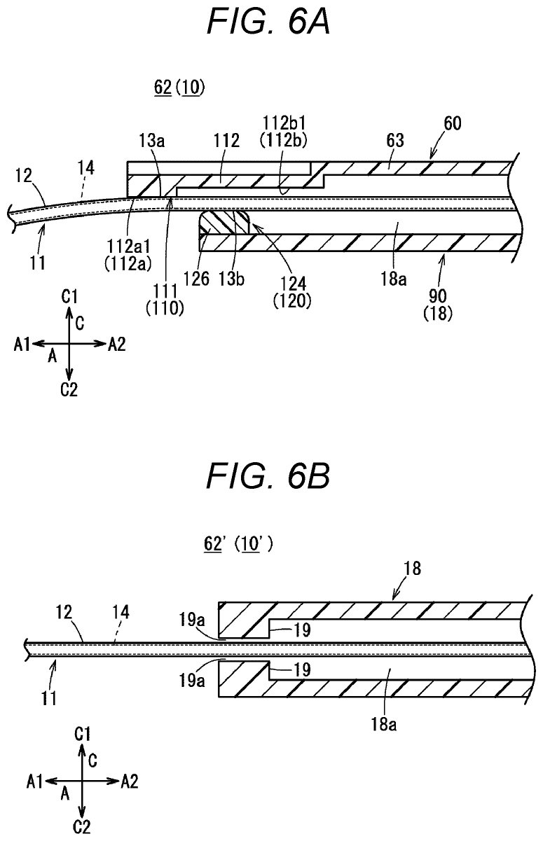

As illustrated in FIGS. 4 and 6A, the support structure portion 62 presses (comes into contact with) offset positions in the extending direction of the catheter 12 to deflect the catheter 12 (multi-structure needle 11) in a side sectional view (when viewed from a direction orthogonal to the extending direction of the catheter 12). Note that FIG. 6A (and subsequent FIGS. 6B to 8, 10A, and 10B) is a side sectional view schematically illustrating the catheter 12 and the support structure portions 62, 62A to 62D.

In the support structure portion 62, the base portion 112 of the upper support portion 111 presses a first position 13a on the upper side of the catheter 12, and the central portion 126 of the lower support portion 124 presses a second position 13b on the lower side of the catheter 12, so that the outer side of the catheter 12 is sandwiched in the arrow C direction. Then, the base portion 112 (first position 13a) presses the catheter 12 on the arrow A1 side with respect to the central portion 126 (second position 13b) of the lower support portion 124. That is, in the base portion 112 having the stepped shape, the distal projecting portion 112a is in contact with the catheter 12, and the proximal extending portion 112b is not in contact with the catheter 12.

Here, in a conventional catheter assembly 10′, a catheter 12 (multi-structure needle 11) is linearly extended along the arrow A direction by an inner needle 14 extending inside the catheter 12 as illustrated in FIG. 6B. Then, a support structure portion 62′ (for example, a support point 19 formed at a distal end of a grip 18) is arranged at a position near an outer peripheral surface of the catheter 12 with a slight clearance 19a. That is, the support structure portion 62′ is configured to be capable of clamping the same position in the extending direction of the catheter 12.

On the other hand, in the support structure portion 62 of the present invention, the lower surface 112a1 of the distal projecting portion 112a is located on the arrow C2 side with respect to a top position of the outer peripheral surface of the catheter 12 extending linearly. Further, an upper surface 126a of the central portion 126 of the lower support member 120 is at the same height position as a bottom position of the outer peripheral surface of the catheter 12. That is, the support space 111a of the support structure portion 62 has a length L1 in the arrow C direction set to be smaller than the outer diameter of the catheter 12 in a front view (see FIG. 5).

In the assembled state, the support structure portion 62 configured as described above intentionally deflects the catheter 12 to the arrow C2 side by the distal projecting portion 112a on the distal side of the grip 18 while suppressing the deflection of the catheter 12 on the arrow C2 side by the lower support portion 124 in the grip 18. Therefore, the support structure portion 62 makes the catheter 12 less likely to deflect toward the arrow C1 side. Note that the support structure portion 62 slightly deflects the catheter 12 to the arrow C2 side, and a curvature radius of the catheter 12 at such a curved portion is sufficiently larger than a length of the catheter 12 in the extending direction (for example, set to three times or more the length of the catheter 12 in the extending direction). Therefore, the user can recognize that the catheter 12 exposed to the distal side of the support structure portion 62 extends in parallel to the extending direction of the grip 18, and can perform a puncture operation or the like without discomfort.

Further, in the support structure portion 62, the first position 13a pressed by the distal projecting portion 112a and the second position 13b pressed by the lower support portion 124 are offset in the axial direction of the catheter 12. Therefore, the support structure portion 62 can suppress the sliding resistance applied from the lower support member 120 to the catheter 12 during an advancing operation of the catheter operation member 60, and can smooth the movement of the catheter 12.

Note that the support structure portion 62 of the catheter assembly 10 is not limited to the above, and may have various configurations. Hereinafter, some modifications of the support structure portion 62 will be described.

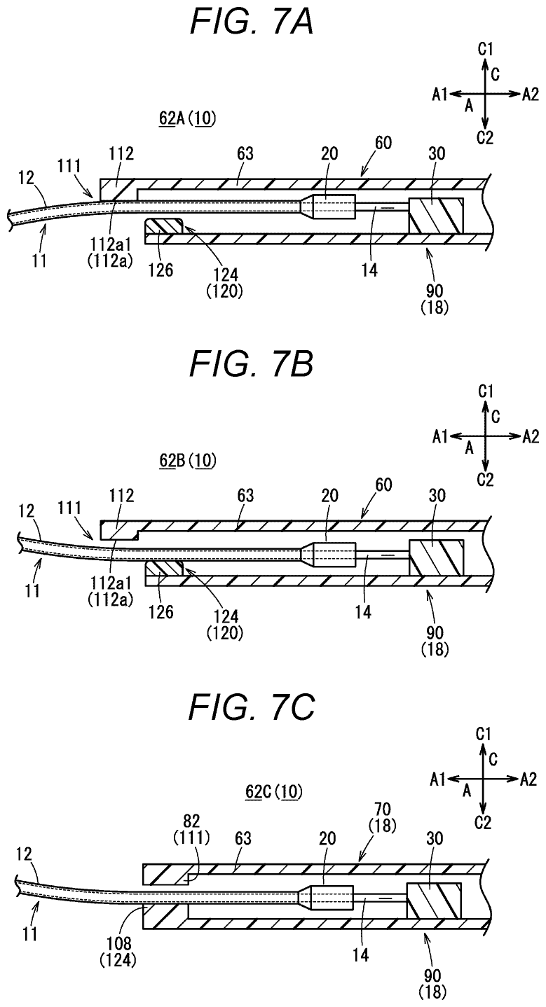

The support structure portion 62A according to a first modification illustrated in FIG. 7A has a configuration in which the upper support portion 111 (distal projecting portion 112a) of the catheter operation member 60 presses the outer peripheral surface of the catheter 12 on the arrow C1 side and the lower support portion 124 is not in contact with the catheter 12 in the assembled state. That is, the catheter 12 is internally supported by the inner needle 14 whose proximal side is fixed by the inner needle hub 30 and is supported to deflect toward the arrow C2 side by the upper support portion 111.

In this manner, even in the configuration in which the catheter 12 is deflected by the support by the inner needle 14 and the pressing by the catheter operation member 60, the support structure portion 62A can firmly support the catheter 12 and stably slide the catheter 12. Then, the lower support portion 124 that is not in contact with the catheter 12 supports the lower side of the catheter 12, for example, when the multi-structure needle 11 is deflected toward the arrow C2 side at the time of puncture. Note that the support structure portion 62A may be configured without the lower support member 120 (lower support portion 124).

The support structure portion 62B according to a second modification illustrated in FIG. 7B has a configuration in which the lower support portion 124 of the lower support member 120 presses the outer peripheral surface of the catheter 12 on the arrow C2 side and the upper support portion 111 is not in contact with the catheter 12 in the assembled state. That is, the catheter 12 is internally supported by the inner needle 14 whose proximal side is fixed by the inner needle hub 30, and is supported by the lower support portion 124 to deflect toward the arrow C1 side on the distal side.

In this manner, even in the configuration in which the catheter 12 is deflected by the support by the inner needle 14 and the pressing by the lower support member 120, the support structure portion 62B can firmly support the catheter 12 and stably slide the catheter 12. The upper support portion 111 that is not in contact with the catheter 12 supports the upper side of the catheter 12, for example, when the multi-structure needle 11 is deflected toward the arrow C1 side at the time of puncture.

The support structure portion 62C according to a third modification illustrated in FIG. 7C does not include the lower support member 120, and a distal portion (protruding portion 108) of the lower grip 90 serves as the lower support portion 124 that comes into direct contact with and presses the catheter 12. As a result, the catheter 12 is internally supported by the inner needle 14 whose proximal side is fixed by the inner needle hub 30, and is supported by the protruding portion 108 to deflect toward the arrow C1 side on the distal side, which is similar to the above-described support structure portion 62B.

In addition, the support structure portion 62C has the upper support portion 111 (a protruding portion 82 of the upper grip 70), which faces the outer peripheral surface of the catheter 12 in a non-contact manner, at a position facing the protruding portion 108 of the lower grip 90. In this manner, the support structure portion 62C that supports the catheter 12 can also be configured using the grip 18. In this case, the catheter 12 and the catheter hub 20 may be operated to move by a catheter operation member (not illustrated) protruding from the inner side to the outer side of the grip 18.

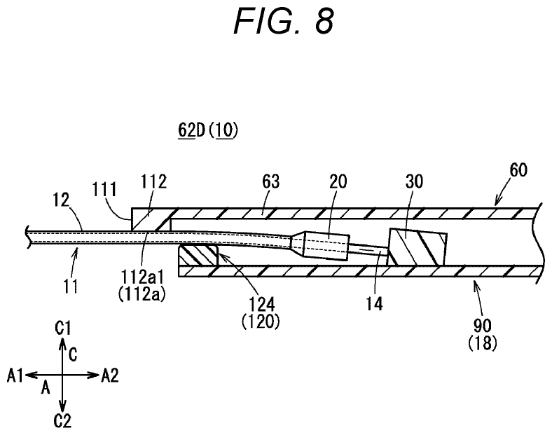

In the support structure portion 62D according to a fourth modification illustrated in FIG. 8, the catheter 12 extends in parallel to the extending direction of the grip 18 (the arrow A direction) on the distal side (the arrow A1 side) of the upper support portion 111 of the catheter operation member 60. Then, the support structure portion 62D deflects the catheter 12 to take a curved posture inside the grip 18. That is, the inner needle hub 30 fixes the inner needle 14 so as to be slightly inclined obliquely upward, and the proximal side of the catheter 12 through which the inner needle 14 is inserted also extends obliquely upward. When being pressed by the upper support portion 111 and the lower support portion 124 (lower support member 120), the catheter 12 is corrected to be elastically deformed (deflected) along the arrow A direction.

Even with the support structure portion 62D configured as described above, it is possible to facilitate the movement of the catheter 12 while firmly supporting the catheter 12 by the support of the upper support portion 111 and the lower support portion 124. In addition, the support structure portion 62D can further enhance the operability at the time of puncture by the user since an exposed portion of the catheter 12 extends in parallel to the extending direction of the grip 18.

The catheter assembly 10 according to the present embodiment is basically configured as described above, and operations thereof will be described hereinafter.

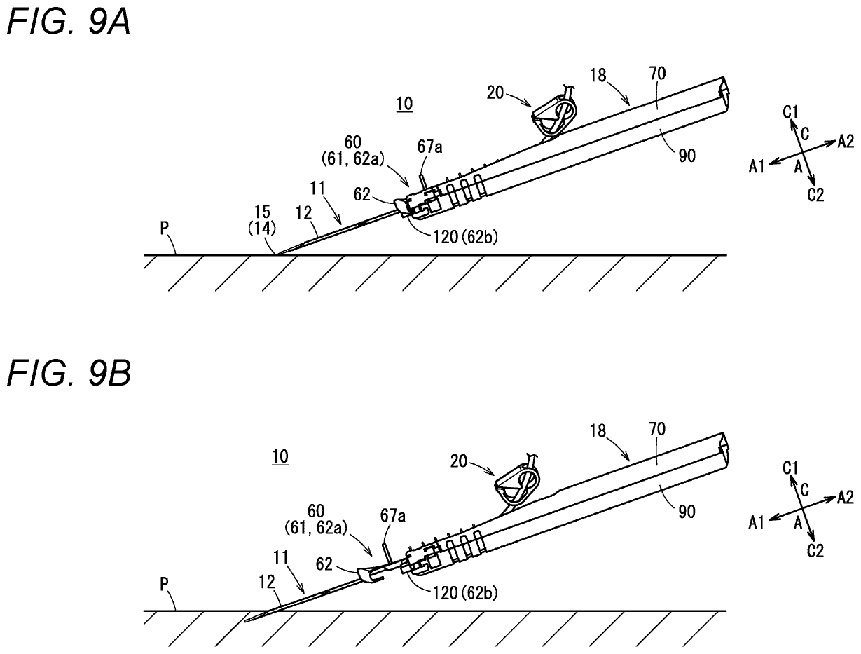

As described above, the catheter assembly 10 is used when infusion, blood transfusion, blood sampling, or the like is performed on the treatment target (living body). As illustrated in FIG. 9A, the user grips and operates the grip 18 to puncture a treatment target P with the multi-structure needle 11 at the time of using the catheter assembly 10.

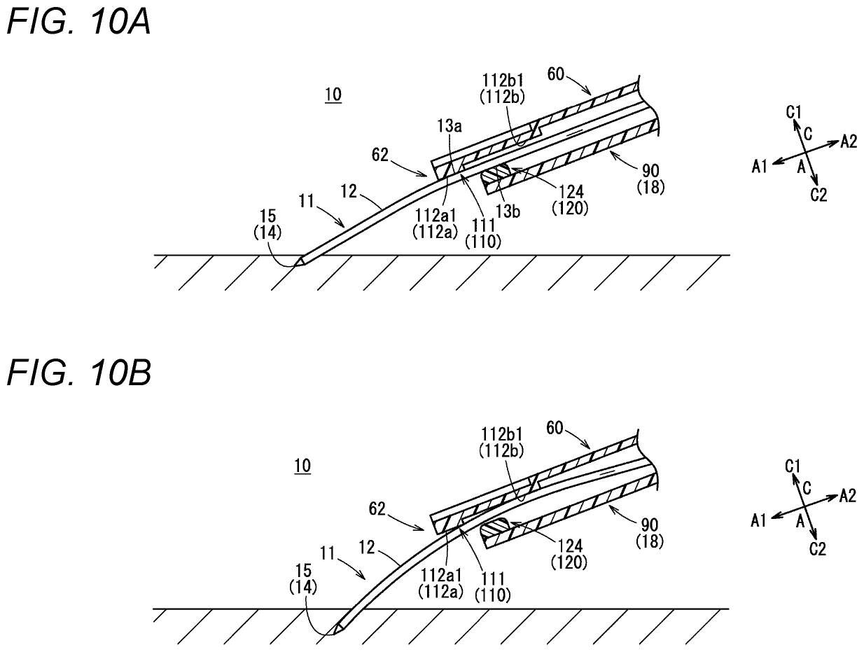

At the time of puncture, the upper support portion 111 of the catheter operation member 60 and the lower support portion 124 of the lower support member 120 press the offset positions (the first position 13a and the second position 13b) in the extending direction of the catheter 12 (multi-structure needle 11) in a side view orthogonal to the extending direction of the catheter 12. The first position 13a and the second position 13b are on opposite sides of the axial center of the catheter 12, and a pressing direction of the catheter 12 connecting the first position 13a and the second position 13b is along the arrow C direction (direction that the blade surface 15a of the inner needle 14 faces). Therefore, as illustrated in FIG. 10A, the support structure portion 62 firmly supports the catheter 12 up to the distal end of the catheter operation member 60, and allows the catheter 12, exposed from the distal end of the catheter operation member 60, to be extended in a state of being deflected in advance toward the arrow C2 side.

As a result, the user can stably insert the multi-structure needle 11 (catheter 12), firmly supported by the support structure portion 62, into the body of the treatment target P. In addition, the pair of distal projecting pieces 113a of the catheter operation member 60 protrudes more greatly toward the arrow C2 side than the upper surface of the lower support portion 124, and thus, the respective distal projecting pieces 113a prevent the catheter 12 from moving in the width direction (arrow B direction) at the time of puncture.

Further, even if the catheter 12 is greatly curved at the time of puncture with the multi-structure needle 11 as illustrated in FIG. 10B, the support structure portion 62 brings the outer peripheral surface of the catheter 12 into contact with the proximal extending portion 112b. That is, the support structure portion 62 comes into contact with a plurality of different points of the catheter 12 in the axial direction when the catheter 12 operates to be deflected more than the assembled state. Therefore, the support structure portion 62 can more effectively suppress the deflection (curvature) of the catheter 12 at the time of puncture.

When the treatment target P is punctured with the multi-structure needle 11 and the needle tip 15 reaches a blood vessel, the user performs an advancing operation of the catheter operation member 60 to advance the catheter 12 beyond the inner needle 14 to be inserted into the blood vessel as illustrated in FIG. 9B. At this time, the catheter operation member 60 advances relatively to the lower support member 120. Further, the support structure portion 62 presses axially offset positions of the catheter 12, thereby reducing the sliding resistance applied from the lower support portion 124. Therefore, the user can smoothly move the catheter 12.

The lower support member 120 is rotatable when a proximal end of the side edge 63a of the catheter operation member 60 (operation plate portion 63) comes out of the grip 18. The lower support member 120 relatively rotates with respect to the grip 18 by coming into contact with and being pushed by the hub engagement portion 64 (wall portion 64b) of the catheter operation member 60 in the advancement process. As a result, members (the catheter hub 20 and the safety member 40) on the proximal side of the hub engagement portion 64 come out of the distal opening portion 18b.

Then, the catheter 12 and the catheter hub 20 are removed from a distal end of the grip 18 by the advancing operation of the user, and then, the safety member 40 protrudes from the distal end of the grip 18. When the catheter operation member 60 is further advanced, the locked protrusion 48 of the safety member 40 moves to the locking portion 100 (an advanced position) of the grip 18. At this time, a distal end of the safety member 40 is exposed from the grip 18 and advances beyond a distal end of the inner needle 14 to cover the inner needle 14, thereby activating an erroneous puncture prevention function.

At the advanced position, the safety member 40 is in a locked state in which movement in both a distal direction and a proximal direction is stopped without coming out of the grip 18. As a result, when the catheter 12, the catheter hub 20, and the catheter operation member 60 are further advanced, the safety member 40 is separated from these members. The engagement between the catheter operation member 60 and the catheter hub 20 can be released as the catheter operation member 60 and the safety member 40 are separated from each other in the catheter assembly 10. Therefore, the catheter 12 and the catheter hub 20 are separated from the lower side of the catheter operation member 60.

The catheter 12 and the catheter hub 20 are detached from the catheter operation member 60 and indwelled in the treatment target P. After the indwelling, a connector (not illustrated) of another medical device is connected to the catheter hub 20. On the other hand, the inner needle 14, the inner needle hub 30, the safety member 40, and the grip 18 in the state of being integrated are appropriately discarded by the user.

Incidentally, the present invention is not limited to the above-described embodiment, and various modifications can be made in accordance with a gist of the invention. For example, the support structure portion 62 or 62D may be configured to press appropriate positions in the circumferential direction without setting the first position 13a and the second position 13b to press the catheter 12 to the opposite positions (positions offset by 180° in phase) in the circumferential direction of the catheter 12.

### Second Embodiment

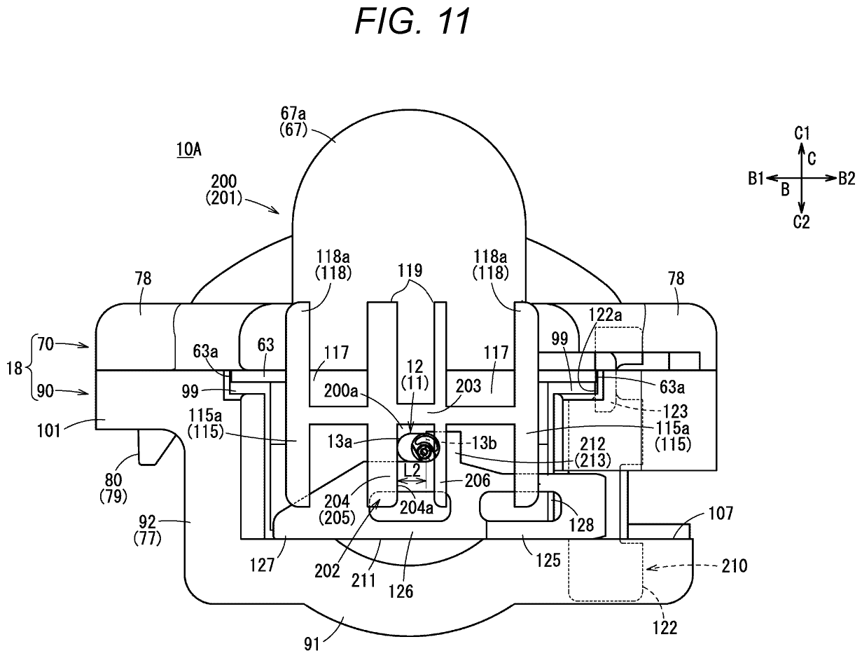

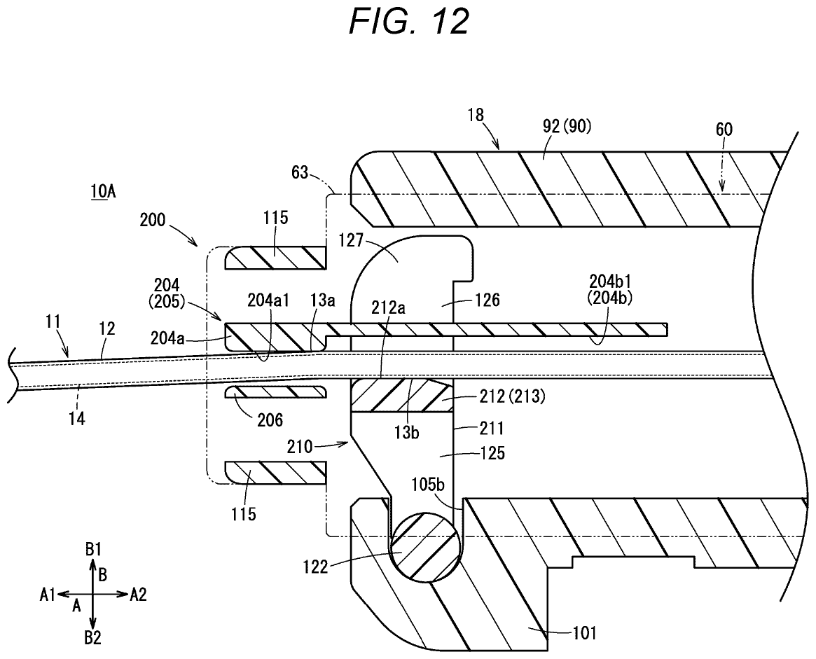

Next, a catheter assembly 10A according to a second embodiment will be described with reference to FIGS. 11 and 12. Incidentally, an element having the same configuration or the same function as that in the above-described embodiment will be denoted by the same reference sign, and the detailed description thereof will be omitted in the following description.

The catheter assembly 10A is different from the above-described catheter assembly 10 that deflects the catheter 12 in the vertical direction (arrow C direction) in terms of including a support structure portion 200 that deflects and supports the catheter 12 (multi-structure needle 11) in the width direction (arrow B direction). That is, the support structure portion 200 includes a catheter operation member 201 and a lower support member 210, and an upper support portion 202 presses the catheter 12 on the arrow B1 side, and a lower support portion 211 presses the catheter 12 on the arrow B2 side.

Specifically, the upper support portion 202 includes a base portion 203 located on the arrow C1 side of the catheter 12, a first ridge portion 204 located on the arrow B1 side of the catheter 12, and a second ridge portion 206 located on the arrow B2 side of the catheter 12. In an assembled state of the catheter assembly 10A, the base portion 203 is not in contact with an outer peripheral surface of the catheter 12 (or is in contact with the outer peripheral surface of the catheter 12 to the extent not to deflect the catheter 12).

On the other hand, the first ridge portion 204 is formed to be thicker than the second ridge portion 206, and serves as a first support portion 205 that presses the outer peripheral surface of the catheter 12. In the first ridge portion 204, a thickness in the width direction on the distal end side is thicker than a thickness in the width direction on the proximal end side, and the inner side (arrow B2 side) facing the second ridge portion 206 has a stepped shape in a plan sectional view. That is, the first ridge portion 204 has, on the arrow B2 side, a distal projecting portion 204a in contact with the outer peripheral surface of the catheter 12 and a proximal extending portion 204b not in contact with the outer peripheral surface of the catheter 12 in the assembled state.

The distal projecting portion 204a is provided in a range overlapping the lower protruding block 115 in a side view, and is formed in substantially the same shape as the lower protruding block 115. A side surface 204a1 of the distal projecting portion 204a on the arrow B2 side is located to be slightly closer to the arrow B1 side than the outer peripheral surface of the catheter 12 supported by the inner needle 14 on the arrow B1 side in the assembled state. On the other hand, the proximal extending portion 204b extends in a range longer than the distal projecting portion 204a in parallel to the extending direction of the catheter 12 (arrow A direction). The proximal extending portion 204b has a side surface 204b1 extending in a non-contact manner at a position near the outer peripheral surface of the catheter 12.

The second ridge portion 206 is also provided in a range overlapping the lower protruding block 115, and is formed in substantially the same shape as the lower protruding block 115. However, the second ridge portion 206 does not have a portion facing the proximal extending portion 204b of the first ridge portion 204. In addition, the reinforcing piece 119 on the arrow B1 side out of the pair of reinforcing pieces 119 is formed to have the same thickness at the same position in the width direction as the first ridge portion 204.

The lower support member 210 is configured such that the lower support portion 211 is arranged on the arrow C2 side of the catheter 12, and the catheter 12 is not pressed by the lower support portion 211, but the catheter 12 is pressed by a projection 212 (second support portion 213) provided to be continuous with the lower support portion 211. The projection 212 protrudes short toward the arrow C1 side at a boundary portion between the coupling portion 125 and the central portion 126 of the lower support portion 211.

A protruding end of the projection 212 is arranged at a position near a lower surface of the base portion 203 of the catheter operation member 201 in the assembled state of the catheter assembly 10A. A side surface 212a of the projection 212 on the arrow B1 side is arranged closer to the arrow B1 side than the side surface of the second ridge portion 206 on the arrow B1 side in the assembled state.

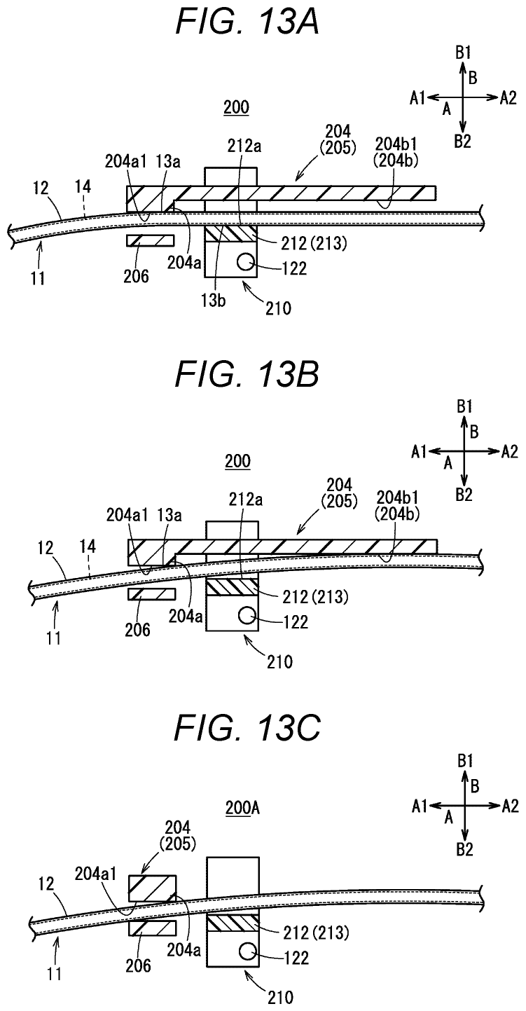

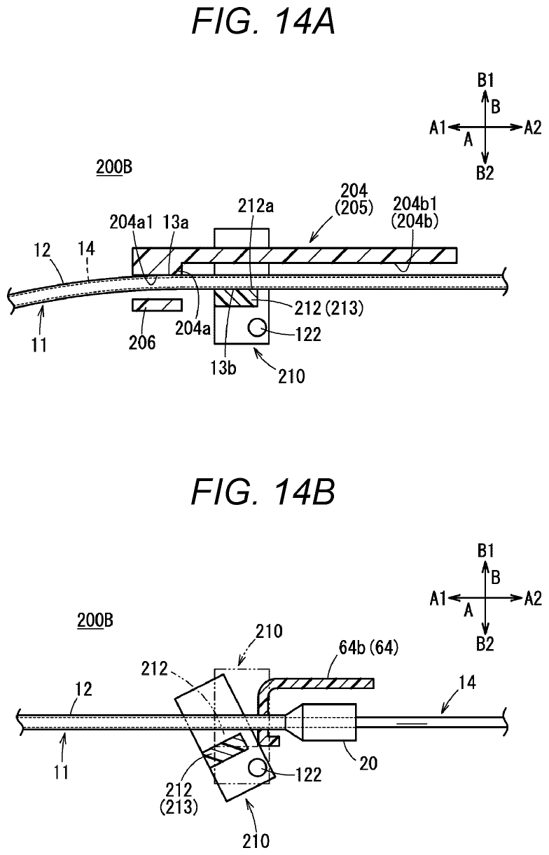

The above-described support structure portion 200 presses (comes into contact with) offset positions in the extending direction of the catheter 12 to deflect the catheter 12 (multi-structure needle 11) in a plan sectional view (when viewed from the direction orthogonal to the extending direction of the catheter 12) as illustrated in FIGS. 12 and 13A. Note that FIG. 13A (and subsequent FIGS. 13B to 14B) is a plan sectional view schematically illustrating the catheter 12 and the support structure portions 200, 200A, and 200B.

That is, in the support structure portion 200, the distal projecting portion 204a presses the first position 13a on the arrow B1 side of the catheter 12, and the projection 212 presses the second position 13b on the arrow B2 side of the catheter 12, so that the outer side of the catheter 12 is sandwiched in the arrow B direction. Then, the distal projecting portion 204a (first position 13a) presses the catheter 12 on the arrow A1 side of the projection 212 (second position 13b). In other words, a support space 200a of the support structure portion 200 has a lateral width (width in the arrow B direction) that is defined by the distal projecting portion 204a and the projection 212 in a front view, and has a length L2 set to be smaller than the outer diameter of the catheter 12.

Therefore, the support structure portion 200 according to the second embodiment can also facilitate the movement of the catheter 12 while firmly supporting the catheter 12 (multi-structure needle 11). Further, even when the catheter 12 is greatly curved at the time of puncture with the multi-structure needle 11 as illustrated in FIG. 13B, the support structure portion 200 can restrict further curvature of the catheter 12 by bringing the outer peripheral surface of the catheter 12 into contact with the proximal extending portion 204b.

Note that the support structure portion 200 can also adopt various modifications. For example, since the inner side of the catheter 12 is supported by the inner needle 14 in the support structure portion 200, one of the configurations (the distal projecting portion 204a and the projection 212) for pressing the catheter 12 at two points may be omitted.

In addition, the support structure portion 200A according to a fifth modification illustrated in FIG. 13C has a configuration in which the second ridge portion 206 has an increased width (thickness) in the arrow B direction. As a result, when the catheter 12 is greatly deflected at the time of puncture, the support structure portion 200A can suppress the deflection of the catheter 12 by the first ridge portion 204 (distal projecting portion 204a) and the second ridge portion 206.

Further, in the support structure portion 200B according to a sixth modification illustrated in FIGS. 14A and 14B, a proximal end (formation range) of the projection 212 on the lower support portion 211 is on the distal side (arrow A1 side) of the axial center of the shaft 122 of the lower support member 210. As a result, the support structure portion 200B suppresses the projection 212 from interfering with the catheter 12 during the rotation of the lower support member 210. That is, the support structure portion 200B can smoothly rotate the lower support member 210 by the contact of the wall portion 64b of the hub engagement portion 64 to favorably deliver the catheter 12 and the catheter hub 20 from the grip 18 when the catheter 12 moves to advance.

### Third Embodiment

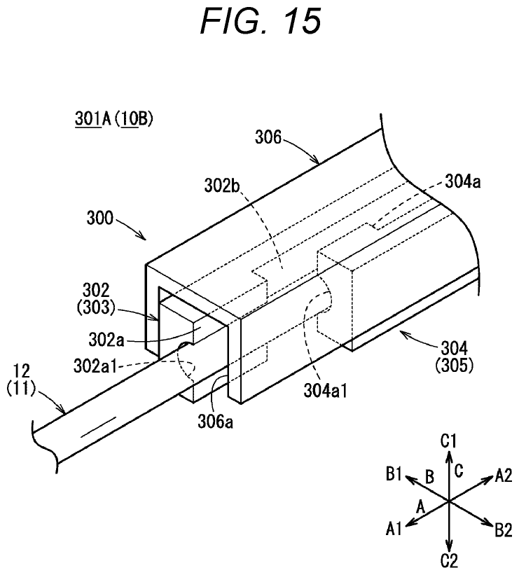

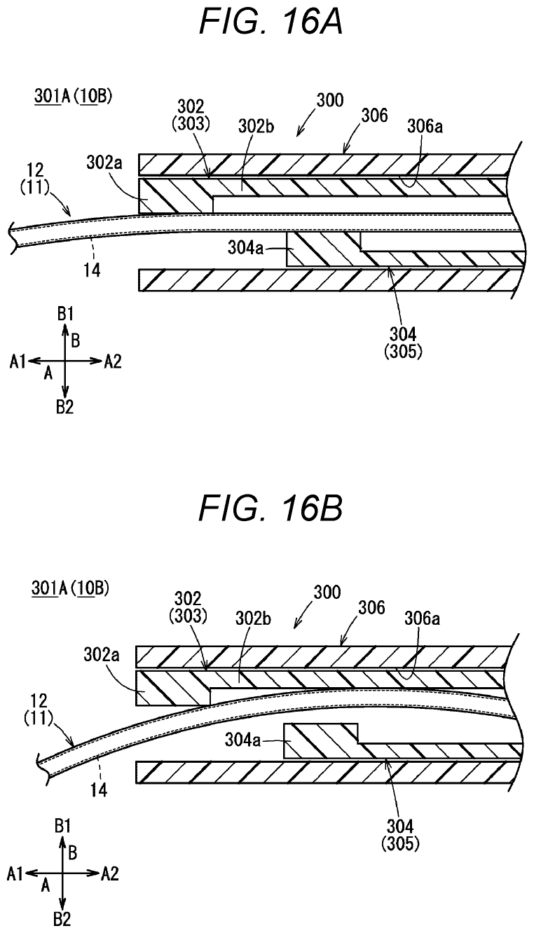

Next, a catheter assembly 10B according to a third embodiment will be described with reference to FIGS. 15 to 16B. The catheter assembly 10B is different from the above-described catheter assembly 10 or 10A in that the catheter 12 is supported at a distal portion of a grip 300 by a support structure portion 301A provided on the grip 300 itself.

Specifically, a distal end of the grip 300 includes a first site 302 (first support portion 303) and a second site 304 (second support portion 305) that can be separated in the width direction (arrow B direction). As illustrated in FIG. 15, the first site 302 has a first lateral projecting portion 302a (distal projecting portion) protruding toward the arrow B2 side at a distal end. A first support groove 302a1 configured to support the catheter 12 is provided on a side surface of the first lateral projecting portion 302a on the arrow B2 side. On the other hand, the second site 304 has a second lateral projecting portion 304a protruding toward the arrow B1 side at a distal end. A second support groove 304a1 configured to support the catheter 12 is provided on a side surface of the second lateral projecting portion 304a on the arrow B1 side.

In addition, the support structure portion 301A includes a sandwiching portion 306 that collectively holds the first and second sites 302 and 304 on the outer side of the first and second sites 302 and 304. The sandwiching portion 306 is formed in a concave shape that is open downward, and has a space portion 306a configured to accommodate the first and second sites 302 and 304 on the inner side. The sandwiching portion 306 constitutes, for example, a part of the grip 300 (a portion covering an upper surface of the grip 300), and moves upward or in the distal direction based on an advancing operation of the catheter 12 and the catheter hub 20 to release the sandwiching of the first and second sites 302 and 304.

Alternatively, the sandwiching portion 306 may also be configured using a member different from the grip 300. For example, the sandwiching portion 306 may be configured using the catheter operation member 60, or may be configured using an operation member configured for a user to operate the blunt needle 50 or a guide wire (not illustrated).

Then, the support structure portion 301A supports the catheter 12 (multi-structure needle 11) to be deflected in the arrow B direction by assembling the first site 302, the second site 304, and the sandwiching portion 306 as illustrated in FIGS. 15 and 16A. Specifically, the first site 302 includes the first lateral projecting portion 302a that protrudes toward the arrow B2 side on the distal side, and a proximal extending portion 302b that extends (in a non-contact manner) at a position near the outer peripheral surface of the catheter 12 on the proximal side of the first lateral projecting portion 302a. Then, the first lateral projecting portion 302a presses the outer peripheral surface (first position 13a) of the catheter 12 on the arrow B1 side, the second lateral projecting portion 304a of the second site 304 presses the outer peripheral surface (second position 13b) of the catheter 12 on the arrow B2 side.

Further, the first lateral projecting portion 302a protrudes to the distal side (arrow A1 side) more than the second lateral projecting portion 304a, and the first position 13a and the second position 13b are offset in the extending direction of the catheter 12. As a result, the first support groove 302a1 and the second support groove 304a1 form a support space (not illustrated) having the length L2 smaller than the outer diameter of the catheter 12 in a front view.

Therefore, the catheter assembly 10B can reduce the sliding resistance during movement of the catheter 12 while firmly supporting the catheter 12 on the distal side of the grip 300 by the support structure portion 301A at the time of puncture. Further, even if the catheter 12 is greatly deflected at the time of puncture as illustrated in FIG. 16B, it is possible to suppress the deflection of the catheter 12 on a side surface of the proximal extending portion 302b on the proximal side of the first lateral projecting portion 302a.

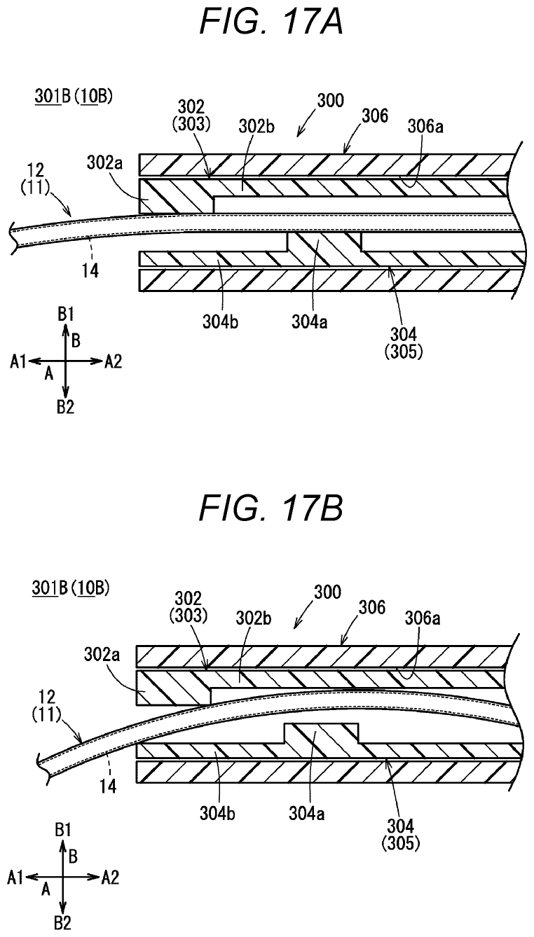

On the other hand, a support structure portion 301B according to a seventh modification illustrated in FIGS. 17A and 17B has a configuration in which a distal extending portion 304b extending to the distal side (arrow A1 side) of the second lateral projecting portion 304a is provided in the second site 304. The distal extending portion 304b extends to the same position as the distal end of the first site 302 and faces the first lateral projecting portion 302a of the first site 302. The distal extending portion 304b is located on the arrow B2 side of the second lateral projecting portion 304a (does not protrude to the arrow B1 side), and forms a relatively large clearance with respect to the first lateral projecting portion 302a. That is, the distal extending portion 304b is not in contact with the catheter 12 in the assembled state.

Since the support structure portion 301B has the distal extending portion 304b in this manner, the catheter 12 comes into contact with a side surface of the distal extending portion 304b (the distal side of the second site 304) when the catheter 12 is greatly deflected at the time of puncture. As a result, the support structure portion 301B can more effectively suppress the deflection of the catheter 12.

Technical ideas and effects that can be grasped from the above-described embodiment are described as follows.

The catheter assembly 10, 10A, or 10B according to an aspect of the present invention includes: the catheter 12; the catheter hub 20 fixed to the catheter 12; the inner needle 14 inserted through the catheter 12; the grip 18 or 300 that fixes and holds the inner needle 14; and the support structure portion 62, 62A to 62D, 200, 200A, 200B, 301A, or 301B that comes into contact with the outer peripheral surface of the catheter 12 to support the outer side of the catheter 12 in the assembled state, and the support structure portion 62, 62A to 62D, 200, 200A, 200B, 301A, or 301B presses the catheter 12 without clamping an identical position in the extending direction of the catheter 12 to deflect the catheter 12 so that the catheter 12 is curved along the extending direction when viewed from the direction orthogonal to the extending direction of the catheter 12 in the assembled state.

According to the above, the catheter assembly 10, 10A, or 10B presses the catheter 12 by the support structure portion 62, 62A to 62D, 200, 200A, 200B, 301A, or 301B to support the catheter 12 to be deflected along the extending direction in the assembled state. As a result, the catheter assembly 10, 10A, or 10B can suppress a deflection of the catheter 12 at the time of puncture. In addition, the support structure portion 62, 62A to 62D, 200, 200A, 200B, 301A, or 301B supports the catheter 12 without clamping the catheter 12, and thus, it is possible to reduce the sliding resistance of the catheter 12 and to easily move the catheter 12 when being operated by the user to move. That is, the catheter assembly 10, 10A, or 10B can greatly improve the operability of the user.

In addition, the support structure portion 62, 62D, 200, 200A, 200B, 301A, or 301B is configured to come into contact with the first position 13a and the second position 13b on the outer peripheral surface of the catheter 12 to support the catheter 12 in the assembled state, and the first position 13a and the second position 13b are on opposite sides of the axial center of the catheter 12, and are offset in the axial direction of the catheter 12. In this manner, the support structure portion 62, 62D, 200, 200A, 200B, 301A, or 301B supports the offset positions in the extending direction of the catheter 12, thereby achieving both suppression of the deflection of the catheter 12 at the time of puncture and facilitation of the movement of the catheter 12.

In addition, in the support structure portion 62, 200, 200A, or 200B, the first support portion 62a or 205 in contact with the first position 13a and the second support portion 62b or 213 in contact with the second position 13b are configured as separate members. As a result, the support structure portion 62, 200, 200A, or 200B can support the catheter 12 by the first support portion 62a or 205 and the second support portion 62b or 213 and easily release the support of the catheter 12 by the relative movement of the first support portion 62a or 205 and the second support portion 62b or 213.

In addition, the first support portion 62a or 205 is the operation unit 61 configured to operate the catheter 12 to move relative to the grip 18, and the second support portion 62b or 213 is the grip 18 or a support member (the lower support member 120 or 210) rotatably attached to the grip 18. As a result, the user can release the support of the catheter 12 and smoothly move the catheter 12 by moving the operation unit 61 relative to the grip 18.

In addition, the first position 13a where the first support portion 62a or 205 is in contact with the catheter 12 is located on the distal side of the second position 13b where the second support portion 62b or 213 is in contact with the catheter 12. As a result, the support structure portion 62, 200, 200A, or 200B can stably move the first support portion 62a or 205 without interfering with the second support portion 62b or 213 at the time of moving the catheter 12.

In addition, the pressing direction of the catheter 12 connecting the first position 13a and the second position 13b is along the direction that the blade surface 15a of the inner needle 14 faces in a front view of the catheter assembly 10. As a result, the support structure portion 62 or 62D can favorably prevent the catheter 12 from being deflected along the direction that the blade surface 15a of the inner needle 14 faces at the time of puncture.

In addition, the pressing direction of the catheter 12 connecting the first position 13a and the second position 13b is orthogonal to the direction that the blade surface 15a of the inner needle 14 faces in a front view of the catheter assembly 10. As a result, the support structure portion 200, 200A, 200B, 301A, or 301B can favorably prevent the catheter 12 from being deflected in the direction orthogonal to the direction that the blade surface 15a of the inner needle 14 faces at the time of puncture.

In addition, the inner side of the catheter 12 is supported by the inner needle 14 extending linearly inside the grip 18, and the support structure portions 62A to 62C press the outer peripheral surface of the catheter 12 from one direction in the assembled state to deflect the catheter 12 along the extending direction. In this manner, the support structure portions 62A to 62C can achieve both suppression of the deflection of the catheter 12 at the time of puncture and facilitation of the movement of the catheter 12 by pressing the outer peripheral surface of the catheter 12 of which the inner side is supported by the inner needle 14 from one direction.

In addition, the support structure portion 62, 62D, 200, 200A, 200B, 301A, or 301B is formed in a stepped shape having the distal projecting portion 112a or 204a (and first lateral projecting portion 302a) that presses the outer peripheral surface of the catheter 12 in the assembled state, and the proximal extending portion 112b, 204b, or 302b that extends in a non-contact manner at a position near the outer peripheral surface of the catheter 12 on the proximal side of the distal projecting portion 112a or 204a (and the first lateral projecting portion 302a). As a result, the support structure portion 62, 62D, 200, 200A, 200B, 301A, or 301B can suppress the deflection of the catheter 12 by the proximal extending portion 112b, 204b, or 302b even when the catheter 12 is operated to be greatly deflected at the time of puncture.

In addition, the support structure portion 301A or 301B includes the sandwiching portion 306 that holds a plurality of sites, which support the outer side of the catheter 12 and are configured to be separable, in a sandwiched state. Since the sandwiching portion 306 is provided in this manner, the support structure portion 301A or 301B can more stably maintain a supporting state of the catheter 12.

What is claimed is:

1. A catheter assembly comprising:
a catheter;
a catheter hub fixed to the catheter;
an inner needle inserted through the catheter;
a grip that fixes and holds the inner needle; and
a support structure portion that contacts an outer peripheral surface of the catheter at a contact position to support an outer side of the catheter in an assembled state;
wherein, in the assembled state, the support structure portion presses the catheter at the contact position without clamping the catheter at the contact position, and thereby deflects the catheter so that the catheter is curved along an extending direction when viewed from a direction orthogonal to the extending direction.

2. The catheter assembly according to claim 1, wherein:
the contact position is a first position and a second position,
the support structure portion contacts the outer peripheral surface of the catheter at the first position and the second position to support the outer side of the catheter in the assembled state, and
the first position and the second position are on opposite sides of an axial center of the catheter and are offset in the extending direction of the catheter.

3. The catheter assembly according to claim 2, wherein:
the support structure portion includes a first support portion in contact with the catheter at the first position and a second support portion in contact with the catheter at the second position, the first support portion and the second support portion being separate members.

4. The catheter assembly according to claim 3, wherein:
the first support portion is an operation unit configured to operate the catheter to move relative to the grip, and
the second support portion is the grip or a support member rotatably attached to the grip.

5. The catheter assembly according to claim 4, wherein:
the first position where the first support portion is in contact with the catheter is located on a distal side of the second position where the second support portion is in contact with the catheter.

6. The catheter assembly according to claim 2, wherein:
a direction connecting the first position and the second position is along a direction that a blade surface of the inner needle faces in a front view of the catheter assembly.

7. The catheter assembly according to claim 2, wherein:
a direction connecting the first position and the second position is orthogonal to a direction that a blade surface of the inner needle faces in a front view of the catheter assembly.

8. The catheter assembly according to claim 1, wherein:
an inner side of the catheter is supported by the inner needle extending linearly inside the grip, and
the support structure portion presses the outer peripheral surface of the catheter from one direction in the assembled state to deflect the catheter so that the catheter is curved along the extending direction.

9. The catheter assembly according to claim 1, wherein:
the support structure portion is formed in a stepped shape having a distal projecting portion that presses the outer peripheral surface of the catheter in the assembled state, and a proximal extending portion that extends in a non-contact manner at a position near the outer peripheral surface of the catheter at a location proximal of the distal projecting portion.

10. The catheter assembly according to claim 1, wherein:
the support structure portion has a sandwiching portion that, in a sandwiched state, holds a plurality of support portions that support the outer side of the catheter and are configured to be separable.

11. A catheter assembly comprising:
a catheter;
a catheter hub fixed to the catheter;
an inner needle inserted through the catheter;
a grip that fixes and holds the inner needle; and
a catheter operation member configured to operate the catheter to move relative to the grip, the catheter operation member comprising a distal projecting portion that protrudes downward and contacts an outer peripheral surface of the catheter at a contact position to support an outer side of the catheter in an assembled state;
wherein, in the assembled state, the distal projecting portion presses the catheter at the contact position without clamping the catheter at the contact position, and thereby deflects the catheter so that the catheter is curved along an extending direction when viewed from a direction orthogonal to the extending direction.

12. A catheter assembly comprising:
a catheter;
a catheter hub fixed to the catheter;
an inner needle inserted through the catheter;
a grip that fixes and holds the inner needle; and
a lower support member rotatably attached to the grip, wherein the lower support member contacts an outer peripheral surface of the catheter at a contact position to support an outer side of the catheter in an assembled state;
wherein, in the assembled state, the lower support member presses the catheter at the contact position without clamping the catheter at the contact position, and thereby deflects the catheter so that the catheter is curved along an extending direction when viewed from a direction orthogonal to the extending direction.
